(19) United States
(12) Patent Application Publication
(10) Pub. No.: US 2024/0360396 A1
(43) Pub. Date: Oct. 31, 2024

(54) MICROSTRUCTURED SCAFFOLD FOR THE PREPARATION OF A TISSUE IMPLANT

(71) Applicants: CENTRE D'ETUDE DES CELLULES SOUCHES (CECS), Corbeil-Essonnes (FR); UNIVERSITE D'EVRY VAL D'ESSONNE, Evry-Courcouronnes (FR); CENTRE NATIONAL DE LA RECHERCHE SCIENTIFIQUE, Paris (FR); UNIVERSITÉ PARIS-SACLAY, Gif sur Yvette (FR)

(72) Inventors: Christelle MONVILLE, Champigny (FR); Elise HERARDOT, Maisons-Alfort (FR); Karim BEN M'BAREK, Créteil (FR); Frédéric HAMOUDA, Savigny sur Orge (FR)

(73) Assignees: CENTRE D'ETUDE DES CELLULES SOUCHES, Corbeil-Essonnes (FR); UNIVERSITE D'EVRY VAL D'ESSONNE, Evry-Courcouronnes (FR); CENTRE NATIONAL DE LA RECHERCHE SCIENTIFIQUE, Paris (FR); UNIVERSITÉ PARIS-SACLAY, Gif sur Yvette (FR)

(21) Appl. No.: 18/580,848

(22) PCT Filed: Jul. 21, 2022
(86) PCT No.: PCT/EP2022/070523

§ 371 Date: Jan. 19, 2024

(30) Foreign Application Priority Data
Jul. 22, 2021 (FR) FR2107934

(51) Int. Cl. C12M 1/12; A61L 27/38

(52) U.S. Cl. CPC C12M 25/02; A61L 27/3813

(57) ABSTRACT

The preparation of a microstructured scaffold corresponding to a biocompatible membrane-type support material for cell culture, and a composition that includes the microstructured scaffold and cells, in particular photoreceptors. Also, the preparation of an implantable graft for the treatment of diseases of the retinal pigment epithelium and the neural retina.

The present invention relates to the preparation of a microstructured scaffold corresponding to a biocompatible membrane-type support material for cell culture. The present invention also relates to a composition comprising the microstructured scaffold and cells, in particular photoreceptors, with or without retinal pigment epithelium cells. The present invention applies in particular to the preparation of an implantable graft for the treatment of diseases of the retinal pigment epithelium and the neural retina.

The retina is the sensory layer that lines the back of the eye, receives the image formed by the lens, and translates this image into neuronal impulses that are transmitted to the brain via the optic nerve. The retina is a neuronal tissue that interacts critically with the retinal pigment epithelium, which is necessary for its survival and proper functioning. The macula, the central part of the retina, is enriched with photoreceptors capable of perceiving different colours and fine visual details. The macula is crucial for facial recognition and reading.

A number of diseases can affect the retinal pigment epithelium or neural retina, resulting in impaired vision that can lead to blindness. These diseases include hereditary or age-related macular degeneration, macular dystrophy and retinitis pigmentosa. The retinal epithelium or neural retina can also be damaged by trauma or infection. Recent research seems to show that retinal lesions can be delayed, stopped or even repaired by transplanting retinal pigment epithelium cells or retinal cells into the subretinal space, which can prevent the reduction in visual capacity caused by these diseases or improve them in the case of transplants of neural retinal cells.

Various forms of transplants or transplantation techniques have been proposed in an attempt to preserve the retina. More recently, promising results have been obtained by transplanting a culture of retinal pigment epithelium cells obtained by differentiation of pluripotent stem cells, the cells being cultured on a biocompatible membrane.

In the case of advanced retinal disease, transplantation of cells from the retinal pigment epithelium alone is not sufficient. The photoreceptors must also be replaced. Photoreceptors are a highly polarised and specialised cell type with apical outer segments containing light-sensitive photopigments and basal axonal terminations.

Transplantation of polarised photoreceptors with or without retinal pigment epithelium cells presents significant challenges due to the need to provide a multilayer graft with an organised structure.

Three-dimensional support microfabrication technology offers potential solutions to these problems.

The manufacture of a scaffolding system for photoreceptor cell culture has already been proposed.

For example, WO2017164992 describes a microstructured scaffold system which comprises a support member comprising micropores allowing photoreceptors to grow in a vertical direction.

The micropores of the microstructured scaffold are made of a spherical or conical part called a “curvilinear cell receiver” connected to at least one cylindrical part called a “cell guide channel”.

The density of the micropores of the support element is a very important parameter, as it conditions the number of possible connections between the photoreceptors and the cells of the retinal epithelium. In the case of WO2017164992, the distance between 2 adjacent micropores is 0.1-5 μm (edge to edge) or 16 μm centre to centre. This scaffold is conducive to numerous intercellular connections. However, the depth of the pores is not conducive to optimal maturation of the photoreceptors. A depth of around 100 μm (corresponding to the size of a mature human photoreceptor) is desired for this type of cell. However, the production of microstructured membranes with a thickness of around 100 μm and a pore periodicity similar to that presented in document WO2017164992 would not be compatible with current manufacturing processes. There is a risk of tearing the patterns (of the present invention) from the master mould when the membrane is demoulded because the period is less than the height of the patterns (50 μm versus 100-120 μm). However, the production of this type of scaffolding can be problematic due to the significant physical constraints of manufacture, in particular the risk of tearing the microstructured membrane when it is demoulded.

It can also be pointed out that the state of the art describes numerous microstructured scaffolds for culturing cells, in particular photoreceptors comprising cylindrically shaped micropores. When a scaffold of this type is seeded with mature or immature photoreceptors, some of the cells fall to the bottom of the micropores, which is problematic.

Photoreceptors are polarised and oriented cells with external segments (photoreceptor elongation) that form contacts, basally, with cells of the retinal pigment epithelium and, apically, with synapses in in vivo tissue. Without suitable culture media and appropriate signalling cues, the correct orientation of photoreceptors in in vitro reconstructed tissue may be difficult to achieve. To overcome this, correct maturation is guided through a specially designed microstructured polymer scaffold with micropores in which immature photoreceptors can be seeded while their orientation is imposed by the microstructure of such a scaffold. The microstructured scaffold guides photoreceptor maturation by imposing lateral physical constraints within the micropores over a retinal pigment epithelium that provides cues to guide photoreceptor maturation to the expected orientation.

There is therefore a need to prepare new scaffolds for cell culture, which are easier to handle due to manufacturing constraints and which allow optimization of the maturation and organization of photoreceptors within the micropores.

One of the objectives of the invention is therefore to propose a microstructured membrane-type scaffold for cell culture, in particular of photoreceptors, in which the density of the micropores has been optimized both to limit the problems of manufacturing the scaffold and to propose a cell organization that is as close as possible to in vivo retinal tissue.

In a first aspect, the invention therefore relates to a scaffold which comprises or consists of a microstructured membrane provided with a plurality of conical through micropores.

Another aspect of the invention concerns the microstructured scaffold for use as a cell culture support, in particular for photoreceptors.

In a second aspect, the invention concerns a composition comprising the microstructured scaffold and cells, in particular photoreceptors.

Another aspect of the invention concerns a process for preparing a composition comprising the microstructured scaffold and cells, in particular photoreceptors.

In a third aspect, the invention concerns an implantable graft comprising the microstructured scaffold, cells, in particular photoreceptors and retinal pigment epithelium cells cultured on a support.

Another aspect of the invention concerns a method for preparing an implantable graft comprising the microstructured scaffold, cells in particular photoreceptors and retinal pigment epithelium cells cultured on a support.

Another aspect of the invention concerns a process for preparing a microstructured scaffold, in particular by photolithography.

The term “scaffold” is used here to designate a biocompatible support of the microstructured membrane type useful for growing cells, in particular photoreceptors.

The scaffold according to the present invention is a microstructured membrane which comprises conical through micropores. The cone-shaped micropores are either tapered over the entire height of the membrane, or partially tapered over a first part of the micropore (height h1) located in the apical part of the membrane; a second part of the micropore (height h2) located in the basal part of the membrane is formed of at least two, preferably three or four, tapered or substantially tapered channels. These channels are preferably not cylindrical or substantially cylindrical in shape.

### Structure of the Cell Culture Scaffold

In the sense of the present invention, the scaffold comprises or consists of a microstructured membrane which has conical through micropores. The diameter and distribution of the micropores are different depending on the orientation of the membrane. The apical surface of the membrane (1) corresponds to the surface facing upwards. The basal surface of the membrane (2) corresponds to the surface of the membrane facing downwards, i.e. the surface of the membrane which in certain embodiments of the invention is in contact with a layer of cells of the retinal pigment epithelium and comprising micropores of a smaller diameter than those on the apical surface of the membrane.

In a particular aspect of the invention,
the apical surface of the membrane (1) has micropores with a diameter (d1) greater than at least three times, preferably at least four times, the diameter (d2) of the micropores of the basal surface of the membrane (2).

In a particular aspect of the invention, the scaffold comprises or consists of a microstructured membrane comprising conical micropores passing through said membrane at a first surface (1) and at a second surface (2) characterized in that,
the apical surface of the membrane (1) has micropores with a diameter (d1) greater than at least three times, preferably at least four times, the diameter (d2) of the micropores of the basal surface of the membrane (2).

In another particular aspect of the invention, the conical micropore has a first (apical) part of height (h1) and a second (basal) part of height (h2) which has several channels forming several micropores on the basal surface of the membrane (2).

The height (h1) of the micropore is approximately one quarter to one half of the total height of the micropore. Preferably, the height (h1) of the micropore is approximately one third of the total height of the micropore.

The height (h1) of the micropore is comprised from 20 μm to 60 μm, preferably from 30 μm to 50 μm, more preferably from 30 μm to 40 μm, i.e. 30, 31, 32, 33, 34, 35, 36, 37, 38, 39 or 40 μm.

The height (h2) of the micropore is comprised from 40 μm to 80 μm, preferably from 50 μm to 70 μm, more preferably from 55 μm to 65 μm, i.e. 55, 56, 57, 58, 59, 60, 61, 62, 63, 64 or 65 μm.

According to the present invention, when the conical micropore has two channels, the micropore is called bipod; when the conical micropore has three channels, the micropore is called tripod and when the conical micropore has four channels, the micropore is called quadripod.

In a particular aspect of the invention, the conical micropores are of bipod, tripod or quadripod type.

According to the present invention, the conical micropore has a diameter (d1) at the apical surface of the membrane (1) comprised from 15 μm to 25 μm, preferably from 18 μm to 22 μm.

The diameter (d1) of the micropore at the apical surface of the membrane (1) has a value of 15, 16, 17, 18, 19, 20, 21, 22, 23, 24 or 25 μm.

According to the present invention, the cone-shaped micropore has a diameter (d2) at the basal surface of the membrane (2) comprised from 4 μm to 8 μm, preferably from 5 μm to 6 μm.

The diameter (d2) of the micropore at the basal surface of the membrane (2) is 4, 5, 6, 7 or 8 μm.

According to the present invention, the microstructured membrane has a plurality of conical micropores of the bipod, tripod or quadripod type, the micropores being characterized by
a diameter (d1) at the apical surface of the membrane (1) comprised from 15 μm to 25 μm, preferably from 18 μm to 22 μm,
a diameter (d1′) whose value corresponds to the diameter (d1) reduced by 2 to 6 μm, preferably 3 to 5 μm
a diameter (d2) at the basal surface of the membrane (2) comprised from 4 μm to 8 μm, preferably from 5 μm to 6 μm,
a height h1 comprised from 20 μm to 60 μm,
a height h2 comprised from 40 μm to 80 μm, it being understood that the total height (h1+h2) of the microstructured membrane comprised from 80 μm to 120 μm.

According to the present invention, the diameter (d1′) of the bipod, tripod or quadripod conical micropore corresponds to the diameter of the micropore in the area where the conical part of the micropore has several, preferably conical, channels.

According to the present invention, the diameter (d1′) of the conical bipod, tripod or quadripod micropore is comprised from 9 μm to 23 μm, preferably from 16 μm to 20 m.

In a particular aspect of the invention, the scaffold comprises or consists of a microstructured membrane comprising conical micropores passing through said membrane at a first surface (1) and at a second surface (2), characterized in that,
the apical surface of the membrane (1) has micropores with a diameter (d1) comprised from 15 μm to 25 μm, preferably from 18 μm to 22 μm,
the basal surface of the membrane (2) has micropores with a diameter (d2) comprised from 4 μm to 8 μm, preferably from 5 μm to 6 μm.

In a particular aspect of the invention, the thickness of the microstructured membrane is comprised from 80 μm to 120 μm, preferably from 90 μm to 110 μm, even more preferably from 95 μm to 105 μm or even more preferably around 100 μm.

In a particular aspect of the invention, the micropores are spaced by a distance from 20 μm to 40 μm, preferably 30, 31, 32, 33, 34, 35, 36, 37, 38, 39 or 40 μm from edge to edge.

To increase the number of photoreceptor contacts with retinal pigment epithelium cells, the number of micropores on the basal side of the microstructured membrane is increased by a factor of 2, 3 or 4 or more. This technical strategy improves the number of cell interactions without the need to increase the density of micropores as is the case in WO2017164992.

Due to the physical constraints of manufacturing the microstructured membrane, the density of the micropores of the microstructured membrane of the present invention is such that the distance between two adjacent micropores on the apical face of the membrane is at least 20 μm, preferably at least 25 μm, even more preferably at least 30 μm and even more preferably at least 35 μm.

In a particular aspect of the invention, the scaffold consists of a microstructured membrane comprising conical micropores passing through said membrane at a first surface (1) and at a second surface (2), characterized in that,
the apical surface of the membrane (1) has micropores with a diameter (d1) comprised from 18 μm to 22 μm, preferably 20 μm,
the basal surface of the membrane (2) has micropores with a diameter (d2) comprised from 4 μm to 8 μm, preferably 5 μm,
the micropores are spaced by a distance from 20 μm to 40 μm, preferably 35 μm, from edge to edge,
the thickness of the microstructured membrane is comprised from 80 μm to 120 μm, preferably 100 μm.

The scaffold according to the present invention comprises or consists of a microstructured membrane made of a biocompatible, flexible and/or extensible polymer, and in certain particularly suitable embodiments, the polymer is biodegradable.

Suitable polymers include, but are not limited to, synthetic rubbers such as silicone rubbers (e.g. polydimethylsiloxane (PDMS)), polyurethane rubber, styrene butadiene rubber and acrylonitrile butadiene rubber, elastomeric natural rubbers (e.g. thermoplastic polyurethane, thermoplastic copolyester, thermoplastic polyamide), epoxies (e.g. SU-8), polyimides, polyurethanes, polyamides, polyesters (e.g. poly(lactic-co-glycolic acid) (PLGA), polylactic acid (PLA), polyglycolic acid (PGA), polycaprolactone (PCL), poly(glycerol sebacate) (PGS)), polysaccharides (e.g. chitosan), parylene, and combinations thereof.

Advantageously, these polymers are of the “plastic type”, which makes the scaffolding easier to handle.

In one particular aspect, the polymer is PDMS or PLGA.

Another aspect of the invention concerns a composition comprising the microstructured scaffold and cells, in particular photoreceptors.

In a particular embodiment of the invention, the seeded cells are in particular photoreceptors, photoreceptor precursors or retinal progenitors.

In a particular embodiment of the invention, the scaffold is characterized in that the microstructured membrane is seeded with cells, in particular photoreceptors, photoreceptor precursors or retinal progenitors.

The present composition useful for cell therapy to treat damaged tissue comprises:
the microstructured membrane as described above, and
at least one photoreceptor, photoreceptor precursor or retinal progenitor per micropore.

The micropores of the microstructured membrane are configured to allow adhesion of at least one photoreceptor (or photoreceptor precursor or retinal progenitor) to the inner wall of a micropore.

The micropores of the microstructured membrane are configured to allow photoreceptor precursors or retinal progenitors to differentiate into photoreceptors and/or mature.

The microstructured scaffold can be used to promote maturation, survival, differentiation, cell attachment and/or cell integration in the host retina by adding various biological products or small molecules. These biological products may be neurotrophins (e.g. CNTF, BDNF, NT-3, NT-4, NGF, GDNF, IGF1) or cytokines (e.g. IL-13, IL-11, IL-10, IL-6, IL-4, IL-1ra, TGF-beta) or small molecules or coating proteins (e.g. collagen, laminin, vitronectin, fibronectin). These biological products can also be extracellular vesicles comprising exosomes, microvesicles, apoptotic bodies which can be derived from various cell types such as pluripotent stem cells, mesenchymal stem cells, differentiated or progenitor cells derived from embryos or foetuses, differentiated cells from adults (primary cells) or cell lines.

These biological products are progressively released during cell culture and/or after transplantation into the host retina. The control of the progressive release of biological products can be induced by the degradation of the polymer (if biodegradable) or by the concentration/density of the polymer or size of the polymer matrix or initial concentration of biological products. In the specific case of a non-degradable polymer, the network created by the polymer encloses the biological products and allows them to be released progressively. The denser the network, the slower the release.

Photoreceptors or photoreceptor precursors or retinal progenitors (which can further differentiate into photoreceptors within the microstructured scaffold) can be seeded into the microstructured scaffold after dissociation and cell counting. These cells can be seeded at a concentration comprised from 10,000 cells per cm² to 1,000,000 per cm². These cells can be differentiated from human pluripotent stem cells (human induced pluripotent stem cells or human embryonic stem cells). The cell population seeded in the microstructured scaffold may be pure or may contain a mixed population of immature and/or mature retinal cells (e.g. bipolar cells, ganglion cells, amacrine cells).

In another embodiment of the invention, the number of cells, in particular photoreceptors, per micropore is from 1 to 20 or more, preferably from 5 to 15, even more preferably from 7 to 12, or approximately 10, i.e. 7, 8, 9, 10, 11 or 12 cells per micropore of the microstructured membrane.

In a particular embodiment of the invention, the micropores of the microstructured membrane are filled with a soft polymer (e.g. hyaluronic acid) or a culture medium containing nutrients and growth factors.

Another aspect of the invention concerns an implantable graft which comprises or consists of an upper layer of photoreceptors or photoreceptor precursors or retinal progenitors seeded inside a microstructured scaffold and a lower layer of retinal pigment epithelium cells cultured on a biological or polymeric support.

Another aspect of the invention concerns the use of an implantable graft as a pharmaceutical composition, said graft comprising or consisting of an upper layer of photoreceptors or photoreceptor precursors or retinal progenitors seeded inside a microstructured scaffold, a lower layer of retinal pigment epithelium cells cultured on a biological or polymeric support.

Another aspect of the invention relates to a composition useful for cell therapy to treat damaged tissue comprising:
the microstructured membrane as described,
at least one photoreceptor (or photoreceptor precursor or retinal progenitor), wherein the micropores are configured to allow adhesion of at least one photoreceptor (or photoreceptor precursor or retinal progenitor) to the inner wall of a micropore, and
a monolayer of retinal pigment epithelium cells,
   in which the at least one photoreceptor is capable of interacting physically or by remote communication via secretion of a biological factor with one or more types of cell located in contact with the first surface (1) of the membrane and at the second surface (2) of the membrane resting on the monolayer of cells of the retinal pigment epithelium.

Retinal pigment epithelium cells can be grown first to form a functional epithelium on top of a thin scaffold that can be made of flexible polymers that can be stretchable and biodegradable (PDMS, PLLA/PLGA, parylene, polyester, polyimide, polyether urethanes, PET membranes, PHA: PHB-HV, ePTFE, PCL, polybutylene, PEGDMA, PTMC) or can be a biological substrate (collagen gel, gelatin gel, fibrin gel, human amniotic membrane, Bruch's membrane, silk fibroin, bacterial cellulose or a membrane made of a biocompatible synthetic material, biodegradable or not). When the retinal pigment epithelium is reconstructed in vitro, the microstructured scaffold is placed on its surface.

According to one embodiment, the cells arranged on the membrane are derived from pluripotent stem cells, multipotent adult stem cells or are derived from primary cultures or cell lines, or correspond to a cell type of the implantation zone, or are simple epithelial cells or cells of different subtypes, such as photoreceptors, ganglion cells, bipolar cells, amacrine cells, retinal pigment epithelium cells and endothelial cells. The human pluripotent stem cells used for the implementation of the invention do not require the destruction of a human embryo and do not have the capacity to induce the development process of a human being.

In one example, the cells in the culture are retinal pigment epithelium cells obtained from human pluripotent stem cells arranged on a denuded amniotic membrane and then cultured for four weeks.

The invention can be applied to implants other than retinal pigment epithelium cell implants. The invention can be applied to the transplantation of retinal cells of various subtypes used alone or in combination (for example, for the retina: photoreceptors, ganglion cells, bipolar cells, amacrine cells, retinal pigment epithelium cells, endothelial cells). These cells can be combined in monolayers or multilayers to reconstruct an artificial retina. They may also be corneal cells or any other type of epithelial cell. More generally, the invention can be applied to any type of epithelium or cell culture in a monolayer on a biological or synthetic membrane.

According to a particular mode of the invention, the microstructured membrane or implant according to the invention has a height comprised from 80 to 120 μm, a length and a width comprised from 2 mm to 10 mm, preferably a height of 120 μm, a length and a width of 5 mm.

According to a particular mode of the invention, the microstructured membrane has a number of pores/mm² comprised from 250 to 1000.

For example, a microstructured membrane according to the invention has 400 pores/mm² i.e 10⁴ pores for 25 mm² with a periodicity of 50 μm or 625 pores/mm² for 25 mm² with a periodicity of 40 μm.

According to a particular mode of the invention, the microstructured membrane comprising tripods has on the basal surface 3600 pores/mm² i.e. 9*10⁴ for 25 mm² with a periodicity of 50 μm or 5625 pores/mm² i.e. 140625 for 25 mm² with a periodicity of 40 μm.

Non-limiting examples of the invention will be described below in relation to the attached figures, including:

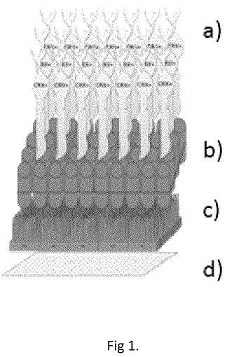

FIG. 1 shows a modified retinal tissue composed of 4 elements comprising (a) an upper layer of photoreceptors seeded inside (b) a microstructured polymer membrane and (c) a lower layer of retinal pigment epithelial cells cultured on (d) a biological or polymer support.

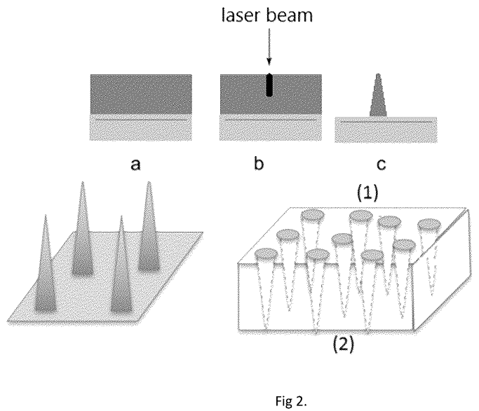

FIG. 2 describes the main steps (a-c) in the preparation of a microstructured membrane of the invention by 2-photon lithography. The faces (1) and (2) of the scaffold correspond to the apical and basal faces respectively.

FIG. 3 (Scanning electron microscopy (SEM) image) shows the cone-shaped structures of a master mould obtained by 2-photon lithography and used to make the cone-shaped scaffolds.

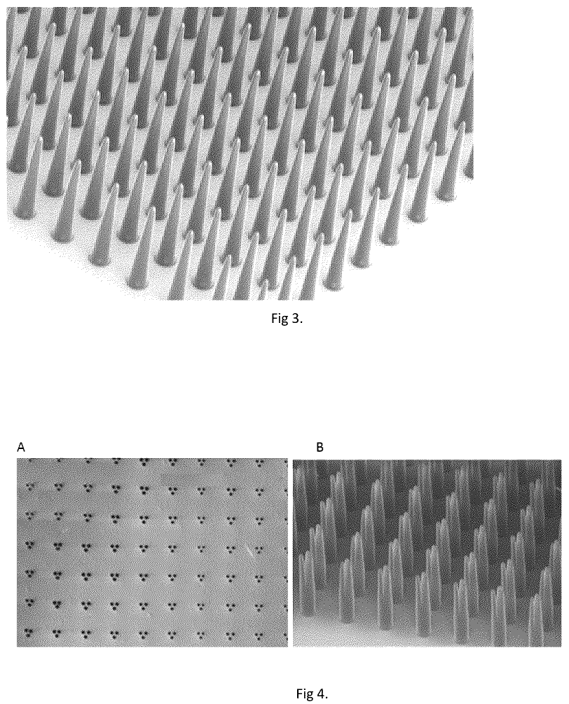

FIG. 4 shows a microstructured membrane (face 2) (4A) obtained after moulding/de-moulding a polymer on a master mould of tripod conical shape (4B) obtained by 2-photon lithography.

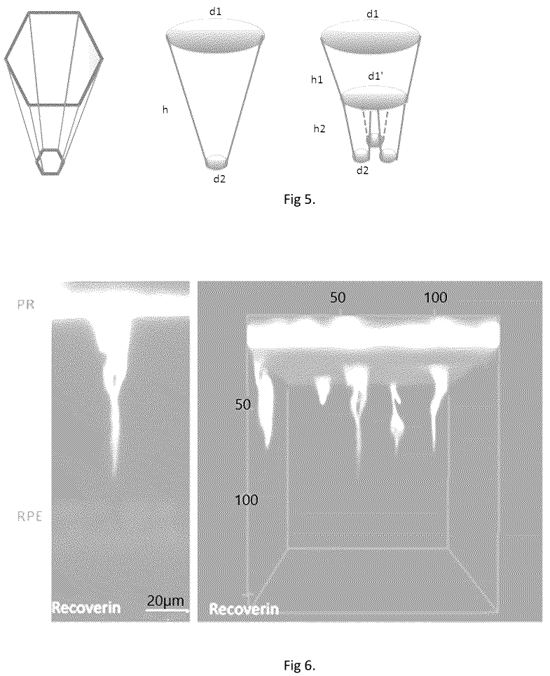

FIG. 5 shows a diagram of a conical micropore, a conical/tripod micropore and a hexagonal conical micropore.

FIG. 6 shows a confocal fluorescence microscopy image of several micropores in a microstructured membrane showing the vertical growth of photoreceptors expressing recoverin and stands on retinal pigment epithelium cells (PR=photoreceptors; RPE: retinal pigment epithelium).

### METHODS FOR PREPARING A PRIMARY SCAFFOLD

The scaffold according to the present invention can be prepared by forming a master mould.

For example, to prepare a primary scaffold, a master mould comprising the negative of the micropores (=micropillars) is prepared using 2-photon lithography, plasma etching or any other technique known to the skilled person.

More typically, the microstructured membrane is moulded onto the master mould, then demoulded and finally the pores are opened using isotropic plasma etching.

The microstructured membrane of the invention is manufactured using mouldable polymers (e.g. PDMS), moulded from the Silicon master mould on which the micropillars are structured with resin, into which the PDMS or other polymer (for the scaffold) is subsequently cast.

Although described herein as the preparation of scaffolds using photolithography, moulding and plasma etching, it should be understood that any means of preparing polymer scaffolds as known in the art may be used without departing from the present invention. Other suitable methods include, for example, direct printing using a 3D printer or moulding using a micro-injection moulding machine.

To prepare the support element, an ultra-thin polymer film is prepared using any process known in the art.

Examples of polymers for use as support elements include those listed above.

Advantageously, the scaffolds of the present invention can be used for cell culture, transplantation, cell development modelling and drug screening.

### 3D Lithography

In laser lithography, two-photon stereolithography (TPS) is used. A focused laser beam scans the volume of a photopolymer point by point to form the desired 3D object. Ultra-short pulses (usually a few hundred femtoseconds) and high confinement of the light wave are used to trigger the biphotonic absorption phenomenon that leads the photoinitiator into an excited state. From this excited state, reactive species can be created to initiate a conventional polymerisation reaction.

A common method of creating microstructured scaffolds is soft lithography using negative tone photoresist, e.g. SU8, to distribute features onto silicon wafers to create masters (SU8-Si) for moulding hard polymer replicas.

FIG. 2 describes the main stages in the formation of the microstructured membrane of the invention.
Step a) involves the preparation of a silicon substrate, the deposition of a resin by centrifugal coating on said substrate, and the heat treatment of the coated substrate.
Step b) involves two-photon laser lithography. The resin polymerises in the areas exposed to radiation from the 2 focused lasers.
Step c) involves the formation of cone-shaped patterns on the silicon substrate obtained after developing the resin. The unexposed areas are soluble in a developer. The master mould is dried using a COr-based supercritical dryer.

The master mould is obtained with conical (and/or multi-pod) patterns with a height of approximately 100 μm. A specific treatment is then applied to the master mould to ensure demoulding.

The polymer is then centrifugally coated onto the master mould. A degassing step removes any air bubbles. A flexible film coated with a water-soluble sacrificial layer is then deposited on the polymer. A force is applied to the film to reduce the residual thickness of the polymer. Annealing is carried out to harden the polymer. Demoulding is achieved by peeling off the flexible film with the PDMS membrane which has been moulded by the master mould patterns.

The membrane is released from the flexible film by dissolving the sacrificial layer.
Plasma etching is carried out to remove the residual thickness of the polymer and create through-apertures in the membrane.

FIG. 3 shows an example of a silicon master mould with a resin microstructured into tapered micropillars, obtained by 2-photon lithography. The 100 μm high micropillars are spaced edge to edge by 35 μm. The image was taken using a scanning electron microscope.

FIG. 4 shows an example (A) of the basal face (2) of a conical tripod scaffold made in PDMS from a silicon master mould (B) coated with a resin microstructured into micropillars 100 μm high and spaced edge to edge by 35 μm. The master mould was obtained using the 2-photon lithography technique.

The process according to the invention is used to produce a microstructured moulded object particularly suitable for the culture of biological cells. The process involves, firstly, a step in which a plastically deformable porous membrane is prepared having a plurality of micropores. The micropores are preferably configured as micropores and are regularly distributed across the film. The micropores are shaped to be continuous from one side of the film to the other, so that they are permeable.

FIG. 5 describes the different examples of micropore patterns used to make microstructured scaffolds. In these examples, the patterns can be simple conical, conical/tripod or conical with hexagonal bases on each face.

### EXAMPLES

### Example 1

### Preparation of a Scaffold

The process of manufacturing a scaffold according to the present invention was initiated by the creation of a master mould from a 2-inch silicon wafer with [100] orientation and a thickness of 280 microns. The wafer was first cleaned with solvents before depositing a photosensitive resin of the SU8 type by centrifugal coating at 3000 rotations per minute (rpm). Heat treatment at 180° C. was carried out after coating to allow evaporation of the solvent contained in the resin.

The 3D 2-photon lithography was carried out on the basis of an STL file previously downloaded to the equipment. This file contains all the tripod patterns over an area of 5 mm×5 mm with a periodicity of 50 μm. After writing the 25 mm² on the resin-coated substrate, the resin was developed in a base and then rinsed in isopropanol. A “critical support” drying process using CO₂ for 45 minutes permanently fixed the 120-micron-high SU8 cone/tripod patterns to the silicon substrate, the whole assembly forming the master mould.

A trimethylchlorosilane-based anti-adhesive treatment lasting 5 minutes was applied to the mould in the vapour phase.
The polymer, PDMS in its liquid state, was mixed with its platinum catalyst in a 13:1 ratio, then degassed and deposited on the master mould by centrifugal coating at 900 rpm for 60 seconds. A 20 μm flexible polycarbonate film coated with a 100 nm sacrificial layer of water-soluble PVA was deposited on the PDMS. A pressure of 190 Pa was applied to reduce the residual thickness of the polymer. A 30 min anneal at 80° C. cured the polymer. The film and membrane were peeled off manually, and the microstructured membrane with 100 micron high tripods was separated from the master mould.
Separation between the membrane and the flexible film was achieved by dissolving the sacrificial layer. The etching of the residual thickness of the microstructured membrane to obtain through holes on each side was carried out in an RIE-type frame with a gas mixture of 02 and CF₄ and a power of 25 W. The etching time depended on the residual thickness, typically between 6 and 7 minutes.

### Example 2

Method for obtaining the tissue composed of photoreceptors and a layer of RPE as shown in FIG. 6

In this example, the tissue composed of photoreceptors and retinal pigment epithelium cells was produced according to the following procedure:

First, human embryonic stem cells were differentiated for 3 weeks into retinal progenitor cells (characterised by expression of the VSX2 and PAX6 genes). Other human embryonic stem cells were then differentiated into retinal pigment epithelium cells for 3 months. These were then frozen in ampoule banks in tanks of liquid nitrogen.
The retinal pigment epithelium cells were then thawed and recultured on a porous plastic medium to form an epithelium for 4 weeks. The culture medium was replaced twice a week. A scaffold with conical micropores (made according to example 1) was sterilised by 70% ethanol baths followed by saline solution baths. The scaffold was coated with a commercial L7 matrix solution (Lonza) diluted 1:100 in a saline solution containing calcium and magnesium, then incubated for 1 hour at 37° C. and 5% carbon dioxide.
The matrix solution was removed after incubation. The scaffold was then placed over the retinal pigment epithelium. Retinal progenitors derived from embryonic stem cells at 3 weeks of culture were then dissociated with Tryple solution, counted and seeded into the scaffold at a density of 500,000 cells per cm². The culture medium was changed 3 times a week for 3 months.
At the end of the 3-month culture period, the tissue was fixed in a 4% paraformaldehyde solution for 15 minutes. The tissue was then rinsed and permeabilised in saline containing 0.1% tritonX100. The tissue was incubated for 30 minutes in the same solution containing 5% animal serum and then exposed overnight to a primary antibody directed against the recoverin protein (expressed by mature photoreceptors). The following day, the tissue was rinsed in a saline solution and then exposed for 1 hour to a secondary antibody directed against the first antibody and coupled to a fluorescent molecule. The tissue was then rinsed, stained with DAPI (to mark the DNA of the cell nucleus) and mounted on microscope slides.
The tissue was imaged using a confocal fluorescence microscope.
FIG. 6 shows, on the left, a reconstruction of a section through the thickness of the tissue where the nuclei of the retinal pigment epithelium cells are visible on the lower part, with photoreceptor extensions just above them, identified by recoverin labelling. The image on the right is a 3-D reconstruction of the tissue in which the recoverin signal is visualised in each of the micropores. The scale is in μm. The recoverin signal is a marker for photoreceptors and shows the vertical polarisation of the cells in the micropores above the retinal pigment epithelium cells.

1.-9. (canceled)

10. A scaffold which comprises or consists of a microstructured membrane comprising conical micropores passing through said membrane at a first apical surface and at a second basal surface wherein the apical surface of the membrane has micropores with a diameter at least three times, preferably at least four times, the diameter of the micropores of the basal surface of the membrane, and
the conical micropores are bipod, tripod or quadripod.

11. The scaffold according to claim 10, wherein:
the apical surface of the membrane has micropores with a diameter comprised from 15 μm to 25 μm,
the basal surface of the membrane has micropores with a diameter comprised from 4 μm to 8 μm.

12. The scaffolding according to claim 10, wherein the thickness of the microstructured membrane is comprised from 80 μm to 120 μm.

13. The scaffolding according to claim 10, wherein the micropores are spaced by a distance from 20 μm to 40 μm from edge to edge.

14. The scaffolding according to claim 10, wherein the microstructured membrane is made of a polymer which is biocompatible.

15. The scaffolding according to claim 14, wherein the polymer is PDMS or PLGA.

16. The scaffolding according to claim 10, wherein the microstructured membrane is seeded with cells in particular photoreceptors, photoreceptor precursors or retinal progenitors.

17. An implantable graft comprising or consisting in:
an upper layer of photoreceptors or photoreceptor precursors or retinal progenitors seeded inside of a microstructured scaffold according to claim 10, and
a lower layer of retinal pigment epithelium cells grown on a biological or polymeric support.
